(19) United States
(12) Patent Application Publication
(10) Pub. No.: US 2025/0068077 A1
(43) Pub. Date: Feb. 27, 2025

(54) CATALYST-FREE CROSSLINKING OF PROPIOLATE-ESTER-FUNCTIONALIZED MOLECULES AND POLYMERS

(71) Applicant: Brewer Science, Inc., Rolla, MO (US)

(72) Inventors: Daniel Patrick Sweat, Rolla, MO (US); Joyce A. Lowes, Rosebud, MO (US)

(21) Appl. No.: 18/813,295

(22) Filed: Aug. 23, 2024

Related U.S. Application Data
(60) Provisional application No. 63/534,246, filed on Aug. 23, 2023.

(51) Int. Cl. G03F 7/11; C07C 67/313; C07C 69/734; C07D 251/32; C08F 8/14; C08F 38/00; C08G 59/14; G03F 7/20

(52) U.S. Cl. CPC G03F 7/11; C07C 67/313; C07C 69/734; C07D 251/32; C08F 8/14; C08F 38/00; C08G 59/1494; G03F 7/2002; C07C 2603/18; C08F 2810/20; C08F 2810/30; C08F 2810/50

(57) ABSTRACT

A novel method to prepare crosslinked thin films without the use of catalysts is disclosed. Propiolic acid is grafted to a glycidyl or epoxy group using a phosphonium catalyst under mild conditions to yield a propiolate ester. The propiolate ester is thermally crosslinkable (and the polymer film rendered insoluble) at temperatures as low as 120° C., and some embodiments may undergo photoinduced crosslinking upon exposure to DUV light. The resulting crosslinked films are equivalent or better in stability to acid-catalyzed epoxide crosslinked films and can be used for a multitude of different applications.

### CROSS-REFERENCE TO RELATED APPLICATIONS

The present application claims the priority benefit of U.S. Provisional Patent Application Ser. No. 63/534,246, filed Aug. 23, 2023, entitled CATALYST-FREE CROSSLINKING OF PROPIOLATE-ESTER-FUNCTIONALIZED MOLECULES AND POLYMERS, the entirety of which is incorporated by reference herein.

### BACKGROUND

### Field

The present disclosure relates to materials and methods for fabricating microelectronic structures.

### Description of Related Art

Crosslinked films are ubiquitous in industrial applications and research and a number of methods have been developed to promote the rapid formation of covalent bonds between polymer chains or small molecules. These can be divided roughly into two categories: a small molecule with multiple functional groups that react with pendant groups on polymer chains, and pendant groups that can react with themselves to form the crosslinked network.

In the first case for thermally crosslinked films, prime examples involve 1,3,4,6-tetrakis(methoxymethyl)glycoluril (TMMGU) and other derivatives that can react with pendant hydroxyl groups in the presence of acid to generate crosslinked networks with the resulting methanol being volatile enough to leave the film. Multiple epoxy groups on a small molecule can also serve as an efficient crosslinker with pendant hydroxyl or phenol groups. Vinyl ethers can react with pendant carboxylic acids and phenols without an additional catalyst. Because these methods all rely on random distribution of a small molecule crosslinker throughout a polymer film, they potentially suffer from inhomogeneity and varying degrees of crosslinking/rigidity of the resulting network. Also, the necessity of acid catalysts requires either a blocked acid to prevent premature crosslinking or the acceptance of defects from the crosslinking reaction while preventing the inclusion of acid-sensitive groups in the crosslinked film.

The second broad category of crosslinking chemistries involves self-reactive groups such as epoxides, which under acid or base catalysis will rapidly generate a crosslinked film. Glycidyl groups especially are common in lithography-relevant thin films due to their stability in storage and ease of reaction (i.e., rapid crosslinking) at moderate temperatures. Poly(benzyl azide)-containing polymers have also shown the ability to crosslink in thin films through loss of nitrogen, but the required temperature of 250° C. limits the degree of other functionality that can be incorporated into the polymer, and the toxicity of azide makes this a less favored route.

Other methods such as thiol-ene reactions or copper-catalyzed alkyne-azide couplings have been used for crosslinking, but generally this crosslinking is either photoinduced or not used for thin films. The necessity of copper as a catalyst also prevents the use of alkyne-azide click couplings in lithography-relevant areas due to metal contamination.

All methods described thus far generally require a catalyst (usually acid) and potentially a small molecule crosslinker, both of which can increase the inhomogeneity of the resulting film and are potential defect generators. Thus, there is a need for improvements in the formation of crosslinked thin films.

### SUMMARY

In one embodiment, a method of forming a structure is provided. The method comprises forming an underlayer on a stack. The underlayer is formed from a composition comprising a compound comprising an acetylenic carboxylic acid reacted with an epoxy group. One or more intermediate layers are optionally formed on the underlayer, with there being an uppermost intermediate layer on the substrate surface, if one or more intermediate layers are present. A photoresist layer is applied to the one or more intermediate layers, if present, or to the underlayer, if no intermediate layers are present.

In a further embodiment, the disclosure provides a structure comprising a substrate having a surface. There are optionally one or more intermediate layers on the substrate surface, there being an uppermost intermediate layer on the substrate surface, if one or more intermediate layers are present. There is an underlayer on the substrate surface, or on the uppermost intermediate layer, if present, and a photoresist on the underlayer. The underlayer comprises a compound comprising an acetylenic carboxylic acid reacted with an epoxy group.

In another embodiment, the disclosure provides a method comprising reacting an acetylenic carboxylic acid with an epoxy group at a temperature of about 100° C. or lower to form a reaction product.

The disclosure further provides a compound having a formula chosen from

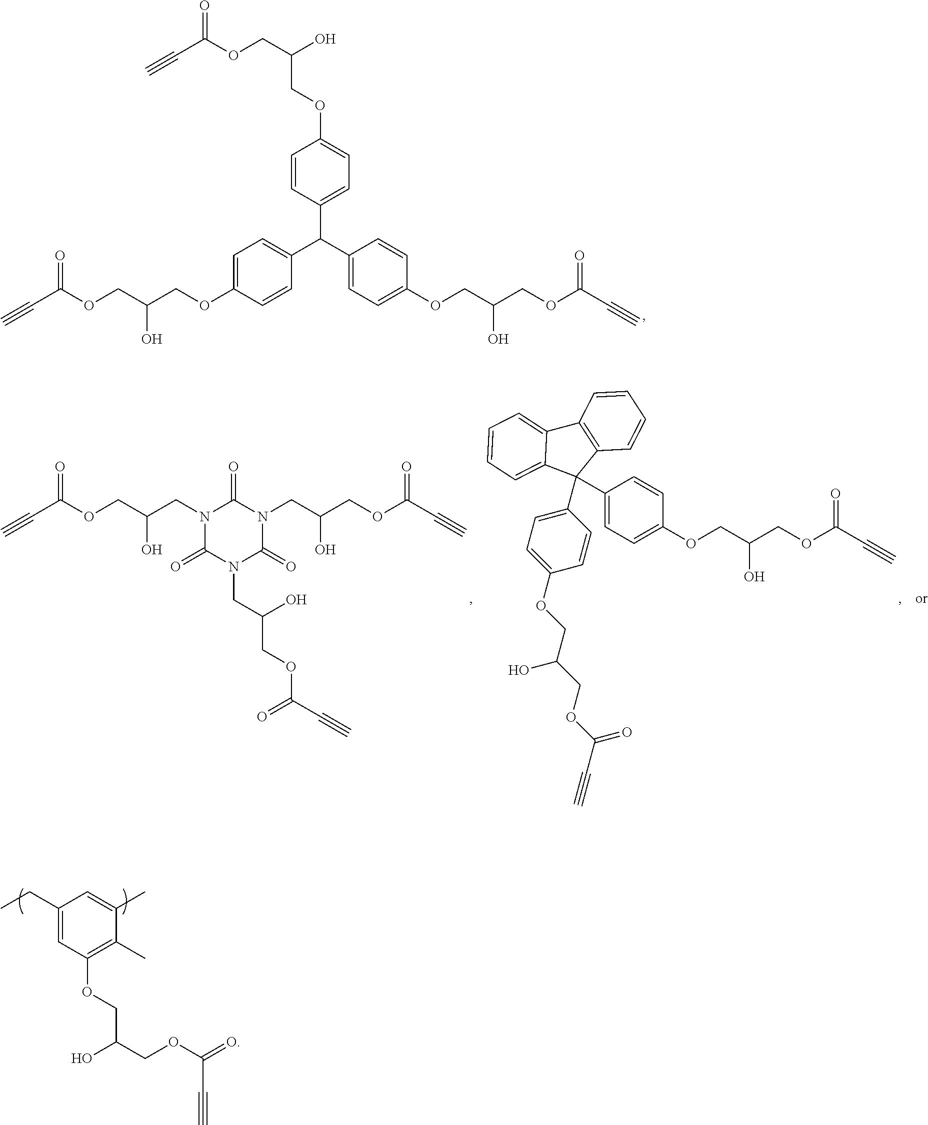

In yet a further embodiment, a crosslinked layer comprising an acetylenic carboxylic acid reacted with an epoxy group is provided.

### BRIEF DESCRIPTION OF THE DRAWINGS

FIG. 1 is a 75-degree tile angle Scanning Electron Microscope (SEM) image of 40/80 nm line/space pattern as generated in Example 31.

FIG. 2 is a 75-degree tile angle SEM image of a 14/28 nm line/space pattern as generated in Example 32.

FIG. 3 is a graph showing thickness before and after stripping film with PGMEA vs. bake temperature for the material from Example 20 as tested in Example 33.

FIG. 4 is a graph showing thickness before and after stripping film with PGMEA vs. bake temperature for the material from Example 21 as tested in Example 33.

FIG. 5 is a graph showing thickness before and after stripping film with PGMEA vs. bake temperature for the material from Example 25 as tested in Example 33.

FIG. 6 is a graph showing thickness before and after stripping film with PGMEA vs. bake temperature for the material from Example 26 as tested in Example 33.

FIG. 7 is a graph showing the ratio of thickness pre- and post-PGMEA strip vs. dose as tested in Example 34.

### DETAILED DESCRIPTION

To address the problems discussed in the background section, a catalyst-free, self-crosslinking technology for crosslinking thin films has been created. Broadly, an acetylenic carboxylic acid is reacted with an epoxy group. For example, in some embodiments, propiolic acid is grafted to a glycidyl or epoxy group on a monomer or polymer to form a propiolate ester. Preferably, the resulting compound comprises the moiety

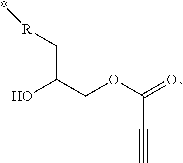

where * represents the attachment point of the moiety to the remainder of the compound, and R is chosen from oxygen, carbon, or nitrogen. It will be appreciated that there could be multiple moieties on the same compound with the same or different R groups (e.g., oxygen, carbon, and/or nitrogen on the same compound) and/or there could be different compounds with the same or different R groups.

The monomer and/or polymer can be used in a variety of thin film-forming compositions, including for use in photolithography. For example, they can be used to make self-crosslinking spin-on-carbon underlayers for lithographic pattern transfer. Moreover, this concept can also be applied to compositions for forming films used for antireflective or adhesion layer functionalities, such as EUV underlayers or reflectivity control in 193-nm photolithography. One example of a general reaction scheme of an acetylenic carboxylic acid reacted with an epoxy group is shown in formula (I).

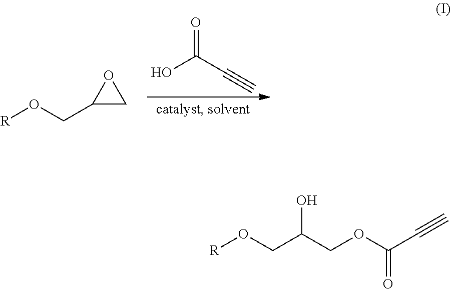

### COMPOSITIONS

### 1. Monomeric Embodiments

Preferred functionalized monomers as described herein are the result of a reaction between an epoxy-containing monomer and an acetylenic carboxylic acid in the presence of catalyst and solvent. In some embodiments, the reaction temperature is about 100° C. or lower to inhibit or prevent the acetylenate ester from self-reacting and gelling during synthesis. This reaction is preferably performed at a temperature of from about 50° C. to about 100° C., more preferably from about 55° C. to about 100° C., and most preferably from about 60° C. to about 75° C., for a time of from about 4 hours to about 36 hours, preferably from about 12 hours to about 30 hours, and more preferably from about 22 hours to about 26 hours.

Suitable acetylenic carboxylic acids include those chosen from C₃ to C₈ acetylenic carboxylic acids, including those chosen from propiolic acid, tetrolic acid, 4-pentynoic acid, 5-hexynoic acid, or mixtures thereof.

Suitable epoxy-containing monomers include those comprising a glycidyl group. In some embodiments, those monomers are chosen from tris-(2,3-epoxy propyl)isocyanurate (TEPIC-S), bisphenol A diglycidyl ether, 9,9-bis(4-glycidyloxyphenyl)fluorene, tris-(4-hydroxyphenyl)methane triglycidyl ether, or combinations thereof.

The acetylenic carboxylic acid and monomer are preferably included at sufficient levels to result in a molar ratio of acetylenic carboxylic acid to epoxy group of about 0.8:1 to about 1.2:1, more preferably about 0.9:1 to about 1.1:1, and even more preferably about 1:1. This typically results in about 3% to about 40% by weight acetylenic carboxylic acid, preferably about 5% to about 35% by weight acetylenic carboxylic acid, and more preferably about 10% to about 30% by weight acetylenic carboxylic acid, and/or about 20% to about 95% by weight epoxy-containing monomer, preferably about 25% to about 85% by weight epoxy-containing monomer, and more preferably about 30% to about 75% by weight epoxy-containing monomer, based on the total weight of solids in the reaction solution taken as 100% by weight.

Suitable catalysts include, but are not limited to, quaternary amine or phosphonium catalysts such as benzyltriethylammonium chloride (BTEAC), ethyltriphenylphosphonium bromide (ETTPB), tetrabutylphosphonium bromide, tetrabutylammonium iodide, or combinations thereof. The catalyst is preferably present in the reaction solution at levels of about 0.5% to about 5% by weight, preferably about 1% to about 3% by weight, and more preferably about 2% to about 2.5% by weight, based on the total weight of solids in the reaction solution taken as 100% by weight.

Preferred reaction solvents include protic solvents or cosolvents because they can limit side reactions. Examples of suitable reaction solvents include those chosen from propylene glycol monomethyl ether (PGME), propylene glycol monomethyl ether acetate (PGMEA), propylene glycol monoethyl ether (PGEE), cyclopentanone, cyclohexanone, anisole, acetophenone, benzyl alcohol, gamma-butyrolactone (GBL), gamma-valerolactone (GVL), tetrahydrofurfuryl alcohol, or mixtures thereof. The reaction has a sufficiently high completion rate such that no isolation of the resulting functionalized monomer is necessary, and the mother liquor can be used for formulations without further preparation or isolation.

The reaction solvent is preferably present in the reaction solution at levels of about 50% to about 80% by weight, preferably about 50% to about 75% by weight, and more preferably about 50% to about 70% by weight, based on the total weight of the reaction solution taken as 100% by weight.

Some embodiments of functionalized monomers are shown in formula II.

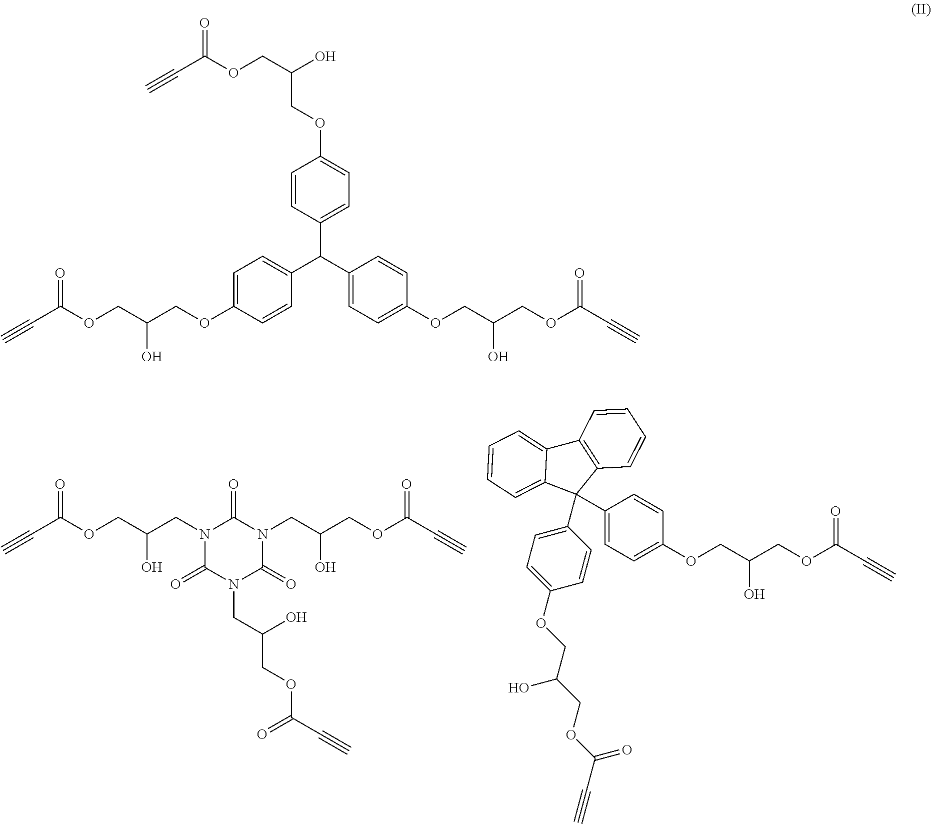

### 2. Polymeric Embodiments

Preferred polymers used for surface modifications as described herein are polymers comprising one or more epoxy-containing monomers, which are then functionalized with an acetylenic carboxylic acid in the presence of catalyst and solvent. Unless stated otherwise, as used herein, the term “polymers” is intended to include oligomers. Oligomers as used herein includes 10 repeat units or fewer.

In some embodiments, the reaction temperature is about 100° C. or lower to inhibit or prevent the acetylenate ester from self-reacting and gelling during synthesis. This reaction is preferably performed at a temperature of from about 50° C. to about 100° C., more preferably from about 55° C. to about 100° C., and most preferably from about 60° C. to about 75° C., for a time of from about 4 hours to about 36 hours, preferably from about 12 hours to about 30 hours, and more preferably from about 22 hours to about 26 hours.

Suitable acetylenic carboxylic acids include those chosen from C₃ to C₈ acetylenic carboxylic acids, including those chosen from propiolic acid, tetrolic acid, 4-pentynoic acid, 5-hexynoic acid, or mixtures thereof.

Suitable polymers comprising one or more epoxy-containing monomers include those polymers comprising one or more monomers comprising a glycidyl group. Suitable polymers include those chosen from epoxy cresol novolac (ECN), poly(glycidyl methacrylate) (poly(GMA)), epoxy phenyl novolac (EPON), poly(4-glycidyloxystyrene), bisphenol A novolac epoxy, poly(glycidyl acrylate), or combinations thereof.

The acetylenic carboxylic acid and polymer are preferably included at sufficient levels to result in a molar ratio of acetylenic carboxylic acid to epoxy group of about 0.8:1 to about 1.2:1, more preferably about 0.9:1 to about 1.1:1, and even more preferably about 1:1. This typically results in about 3% to about 40% by weight acetylenic carboxylic acid, preferably about 5% to about 35% by weight acetylenic carboxylic acid, and more preferably about 10% to about 30% by weight acetylenic carboxylic acid, and/or about 60% to about 95% by weight polymer, preferably about 65% to about 90% by weight polymer, and more preferably about 70% to about 80% by weight polymer, based on the total weight of solids in the reaction solution taken as 100% by weight.

In some embodiments, an epoxy-containing monomer is copolymerized with one or more comonomers. Suitable comonomers include those chosen from styrene, methyl methacrylate, benzyl methacrylate, 4-methylstyrene, pentafluorostyrene, butyl methacrylate, 2-isopropyl-2-adamantylmethacrylate, cyclohexyl methacrylate, 1-adamantylmethacrylate, 4-tert-butoxycarbonyloxystyrene, or combinations thereof. When a comonomer is included, it is typically included at a molar ratio of comonomer to epoxy-containing monomer of about 0.2:1 to about 9:1, preferably about 0.5:1 to about 8:1, more preferably about 0.5:1 to about 8:1, and even more preferably about 0.5:1 to about 6:1.

Suitable catalysts include, but are not limited to, quaternary amine or phosphonium catalysts such as benzyltriethylammonium chloride (BTEAC), ethyltriphenylphosphonium bromide (ETTPB), tetrabutylphosphonium bromide, tetrabutylammonium iodide, or combinations thereof. The catalyst is preferably present in the reaction solution at levels of about 0.1% to about 5% by weight, preferably about 0.5% to about 3% by weight, and more preferably about 1.5% to about 2.5% by weight, based on the total weight of solids in the reaction solution taken as 100% by weight.

Preferred reaction solvents include protic solvents or cosolvents because they can limit side reactions. Examples of suitable reaction solvents include those chosen from PGME, PGMEA, PGEE, cyclopentanone, cyclohexanone, anisole, acetophenone, benzyl alcohol, GBL, GVL, tetrahydrofurfuryl alcohol, or mixtures thereof. The reaction has a sufficiently high completion rate such that no isolation of the resulting functionalized polymer is necessary, and the mother liquor can be used for formulations without further preparation or isolation.

The reaction solvent is preferably present in the reaction solution at levels of about 50% to about 90% by weight, preferably about 50% to about 80% by weight, and more preferably about 50% to about 75% by weight, based on the total weight of the reaction solution taken as 100% by weight.

An exemplary polymer formed from functionalized monomers is shown in formula III.

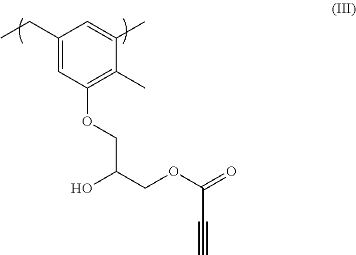

The weight-average molecular weight (Mw) range of the polymers described herein (as measured by gel permeation chromatography) is preferably from about 1,500 g/mol to about 80,000 g/mol, and more preferably from about 4,000 g/mol to about 10,000 g/mol.

In one embodiment, the polymer consists essentially of, or even consists of, one or more types of epoxy-containing monomers. In another embodiment, the polymer consists essentially of, or even consists of, one or more types of epoxy-containing monomers and one or more of the above described comonomers.

### 3. Compositions

The compositions comprise the above-described functionalized polymer and/or above-described functionalized monomer dispersed or dissolved in a solvent system. The combined weight of polymer and/or monomer is preferably utilized at a level of from about 0.1% to about 35% by weight, more preferably from about 0.2% to about 5% by weight, based upon the total weight of the composition taken as 100% by weight.

Preferred solvent systems include one or more solvents chosen from PGME, PGMEA, PGEE, cyclopentanone, cyclohexanone, anisole, GBL, GVL, or mixtures thereof. The solvent system is preferably utilized at a level of from about 65% to about 99.9% by weight, more preferably from about 95% to about 99.8% by weight, based upon the total weight of the composition taken as 100% by weight.

The composition may contain optional ingredients, such as those selected from the group comprising surfactants, polymers, additives, and mixtures thereof. In some embodiments, the compositions comprise less than about 0.1% crosslinker, preferably less than about 0.05% crosslinker, and more preferably about 0% crosslinker, based on the weight of the composition taken as 100% by weight. Additionally or alternatively, the compositions may comprise less than about 0.1% catalyst (e.g., copper catalyst), less than about 0.05% catalyst, and even more preferably about 0% catalyst, based on the weight of the composition taken as 100% by weight.

In some embodiments, the composition comprises less than about 5% by weight, preferably less than about 1%, and more preferably about 0% silicon, based upon the total solids in the composition taken as 100% by weight.

In one or more embodiments, the composition comprises less than about 5% by weight, preferably less than about 1%, and more preferably about 0% nanoparticles, based upon the total solids in the composition taken as 100% by weight.

In some embodiments, the compositions consist essentially of, or even consist of, the functionalized polymer and/or functionalized monomer dispersed or dissolved in a solvent system.

Mixing the above ingredients together in the solvent system forms the particular composition. Furthermore, any optional ingredients (e.g., surfactants) are also suitably dispersed in the solvent system at the same time. The composition is preferably filtered before use, such as with a 0.1-μm or 0.2-μm PTFE filter.

### Methods of Using the Compositions

A method of forming a microelectronic structure that is particularly suited for lithography is provided. Any microelectronic substrate can be utilized, but the substrate is preferably a semiconductor substrate, such as substrates chosen from silicon, SiGe, SiO₂, Si₃N₄, SiON, aluminum, tungsten, tungsten silicide, gallium arsenide, germanium, tantalum, tantalum nitride, Ti₃N₄, hafnium, HfO₂, ruthenium, indium phosphide, tetramethyl silate and tetramethylcyclotetrasiloxane combinations (such as that sold under the name CORAL), SiCOH (such as that sold under the name Black Diamond, by SVM, Santa Clara, CA, US), glass, or combinations of the foregoing. Optional intermediate layers may be formed on the substrate prior to processing, with preferred intermediate layers being TiN or SiO₂ layers. The substrate can have a planar surface, or it can include topographic features (via holes, trenches, contact holes, raised features, lines, etc.). As used herein, “topography” refers to the height or depth of a structure in or on a substrate surface.

The above-described self-crosslinking compositions can be formed into an underlayer, where that underlayer can function as a spin-on carbon layer, an antireflective layer, and/or an adhesion layer (e.g., in EUV applications).

### 1. Inventive Underlayer as Spin-on Carbon Layer

When the above-described composition is used as a spin-on carbon (SOC) layer (also referred to as a carbon-rich layer), a layer of SOC composition is formed on the substrate or any intermediate layers (e.g., a primer layer). The SOC layer can be formed by any known application method, with one preferred method being spin-coating at speeds of about 750 rpm to about 2300 rpm, and preferably about 1200 rpm to about 1800 rpm, for a time period of about 10 seconds to about 60 seconds, and preferably about 15 seconds to about 40 seconds. Preferably, the SOC composition has good spin bowl compatibility, and it will not react or form a precipitate with common photoresist solvents such as PGME, PGMEA, ethyl lactate, cyclohexanone, or mixtures thereof.

After the SOC composition is applied, it is preferably heated to a temperature of about 110° C. to about 250° C., and more preferably about 160° C. to about 180° C., for about 10 seconds to about 120 seconds, and preferably about 30 seconds to about 60 seconds, to evaporate solvents and crosslink the layer.

In another embodiment, the SOC composition can be crosslinked by exposure to radiation. The SOC layer is subsequently fully or partially (e.g., in a pattern) exposed to radiation for a dose of about 10 mJ/cm² to about 2,000 mJ/cm², preferably about 80 mJ/cm² to about 1,000 mJ/cm², and more preferably about 140 mJ/cm² to about 300 mJ/cm² at a wavelength of from about 170 nm to about 365 nm, preferably from about 172 nm to about 365 nm.

The average thickness of the SOC layer after baking is preferably about 1 nm to about 2,000 nm, more preferably about 5 nm to about 300 nm, and even more preferably about 5 nm to about 60 nm. The average thickness is determined by taking the average of thickness measurements at five different locations of the SOC layer, with those thickness measurements being obtained using ellipsometry.

After baking, the SOC layers formed preferably comprise greater than about 65% by weight carbon, more preferably greater than about 80% by weight carbon, and even more preferably about 85% to about 90% by weight carbon, based upon final SOC layer of composition taken as 100% by weight (i.e., the SOC layer is “carbon-rich”).

In one embodiment, the SOC layer has good SC-1 resistance, in that it is not affected by exposure in an SC-1 cleaning solution for at least about 30 minutes at about 60° C.

A hardmask layer may be applied to the SOC layer or to any intermediate layers that might be present on the SOC layer. The hardmask layer can be formed by any known application method, such as chemical vapor deposition (“CVD”) or plasma-enhanced chemical vapor deposition (“PECVD”). Another preferred method comprises spin-coating at speeds of about 1,000 rpm to about 5,000 rpm, and preferably about 1,250 rpm to about 1,750 rpm, for a time period of about 30 seconds to about 120 seconds, and preferably about 45 seconds to about 75 seconds. Suitable hardmask layers should have a high etch bias relative to underlying layers. Preferred hardmask layers have high-silicon-content materials, preferably at least about 30% by weight silicon, and more preferably from about 35% by weight to about 40% by weight silicon, based on the total weight of the hardmask layer.

Suitable hardmask layers are commercially available and can be formed from a composition comprising a polymer or oligomer (e.g., silanes, siloxanes, silsesquioxanes, silicon oxynitride, silicon nitride, polysilicon, amorphous silicon, or combinations thereof) dissolved or dispersed in a solvent system. Some preferred monomers or polymers for use in the hardmask layer are selected from the group containing phenethyltrimethoxysilane (“PETMS”), 2-(carbomethoxy)ethyltrimethoxysilane (“CMETMS”), tetraethoxysilane (“TEOS”), methyltrimethoxysilane, phenyltrimethoxysilane, methyltrimethoxysilane (“MTMS”), ethyltrimethoxysilane (“ETMS”), (3-glycidyoxypropyl)triethoxysilane, 2-(3,4-epoxycyclohexyl)ethyltrimethyoxysilane (“ECHTMS”), and mixtures thereof. Any optional ingredients (e.g., surfactants, acid catalysts, base catalysts, and/or crosslinkers) are dissolved in the solvent system along with the polymer, monomer, and/or oligomer. Preferred hardmask compositions will preferably have a solids content of about 0.1% to about 70%, more preferably about 0.5% to about 10%, and even more preferably about 0.5% to about 1% by weight, based upon the total weight of the composition taken as 100% by weight.

After the hardmask composition is applied, it is preferably heated to a temperature of about 100° C. to about 300° C., and more preferably about 150° C. to about 250° C., and for a time period of about 30 seconds to about 120 seconds, and preferably about 45 seconds to about 60 seconds, to evaporate solvents. The average thickness (measured by ellipsometry over five locations and averaged) of the hardmask layer after baking is preferably about 5 nm to about 50,000 nm, more preferably about 5 nm to about 1000 nm, and even more preferably about 10 nm to about 30 nm.

Next, a photoresist (i.e., imaging layer) can be applied to the SOC layer, or to any hardmask or other intermediate layer on the SOC layer, to form a photoresist layer. The photoresist layer can be formed by any conventional method, with one preferred method being spin coating the photoresist composition at speeds of about 350 rpm to about 4,000 rpm (preferably about 1,000 rpm to about 2,500 rpm) for a time period of about 10 seconds to about 60 seconds (preferably about 10 seconds to about 30 seconds). The photoresist layer is then optionally post-application baked (“PAB”) at a temperature of at least about 70° C., preferably about 80° C. to about 150° C., and more preferably about 100° C. to about 150° C., for time periods of about 30 seconds to about 120 seconds. The average thickness (determined as described previously) of the photoresist layer after baking will typically be about 5 nm to about 120 nm, preferably about 10 nm to about 50 nm, and more preferably about 20 nm to about 40 nm.

The photoresist layer is subsequently patterned by exposure to radiation for a dose of about 10 mJ/cm² to about 200 mJ/cm², preferably about 15 mJ/cm² to about 100 mJ/cm², and more preferably about 20 mJ/cm² to about 80 mJ/cm² at a wavelength of from about 13.5 nm to about 365 nm, preferably from about 13.5 nm to about 248 nm. Suitable wavelengths include DUV, i-line, ArF, KrF, and EUV wavelengths. More specifically, the photoresist layer is exposed using a mask positioned above the surface of the photoresist layer. The mask has areas designed to permit the radiation to reflect from or pass through the mask and contact the surface of the photoresist layer. The remaining portions of the mask are designed to absorb the light to prevent the radiation from contacting the surface of the photoresist layer in certain areas. Those skilled in the art will readily understand that the arrangement of reflecting and absorbing portions is designed based upon the desired pattern to be formed in the photoresist layer and ultimately in the substrate or any intermediate layers.

After exposure, the photoresist layer is preferably subjected to a post-exposure bake (“PEB”) at a temperature of less than about 180° C., preferably about 60° C. to about 140° C., and more preferably about 80° C. to about 130° C., for a time period of about 30 seconds to about 120 seconds (preferably about 30 seconds to about 90 seconds).

The photoresist layer is then contacted with a developer to form the pattern. Depending upon whether the photoresist used is positive-working or negative-working, the developer will either remove the exposed portions of the photoresist layer or remove the unexposed portions of the photoresist layer to form the pattern. The pattern is then transferred through the various layers, and finally to the substrate. This pattern transfer can take place via plasma etching (e.g., CF₄ etchant, 02 etchant) or a wet etching or developing process.

### 2. Inventive Underlayer as Antireflective Layer, Adhesion Layer, or Assist Layer

When the inventive composition is used as an antireflective layer, adhesion layer, or assist layer, a layer of the inventive composition is formed on the substrate or any intermediate layers (e.g., SOC or hardmask layer).

When an SOC is present as an intermediate layer, regardless of the SOC composition utilized, the SOC layer can be formed by any known application method, with one preferred method being spin-coating at speeds of about 500 rpm to about 3,000 rpm, and preferably about 1,200 rpm to about 2,000 rpm, for a time period of about 10 seconds to about 90 seconds, and preferably about 30 seconds to 60 seconds. Preferably, the SOC composition has good spin bowl compatibility, that is, it will not react or form a precipitate with common photoresist solvents such as PGME, PGMEA, ethyl lactate, cyclohexanone, or mixtures thereof.

After the SOC composition is applied, it is preferably heated to a temperature of about 140° C. to about 250° C., and more preferably about 160° C. to about 220° C., for about 10 seconds to about 120 seconds, and preferably about 30 seconds to about 60 seconds, to evaporate solvents.

The average thickness of the SOC layer after baking is preferably about 50 nm to about 3 μm, more preferably about 100 nm to about 300 nm, and even more preferably about 150 nm to about 200 nm. The average thickness is determined by taking the average of thickness measurements at five different locations of the SOC layer, with those thickness measurements being obtained using ellipsometry.

After baking, the SOC layers formed preferably comprise greater than about 75% by weight carbon, more preferably greater than about 80% by weight carbon, and even more preferably about 85% to about 90% by weight carbon, based upon final layer of composition taken as 100% by weight (i.e., the SOC layer is “carbon-rich”).

The SOC layer preferably has little or no shrinkage. That is, the average thickness decreases by less than about 5% after being heated to about 200° C. for about 10 minutes and, even more preferably, the thickness decreases less than about 5% after being heated to about 300° C. for about ten minutes. In some cases, the shrinkage of the SOC layer may be negative, meaning the thickness of the layer increases after the described baking conditions, indicating swelling of the SOC layer. (In these cases, it is theorized that the SOC layer may become less dense after high-temperature bakes, leading to a small weight loss, but slight film swelling.) In one embodiment, the SOC layer has good SC-1 resistance, in that it is not affected by exposure in an SC-1 cleaning solution for more than about 30 minutes at about 60° C.

A hardmask layer may be applied to the SOC layer or to any intermediate layers that might be present on the SOC layer. The hardmask layer can be formed by any known application method, such as chemical vapor deposition (“CVD”) or plasma-enhanced chemical vapor deposition (“PECVD”). Another preferred method comprises spin-coating at speeds of about 1,000 rpm to about 5,000 rpm, and preferably about 1,250 rpm to about 1,750 rpm, for a time period of about 30 seconds to about 120 seconds, and preferably about 45 seconds to about 75 seconds. Suitable hardmask layers should have a high etch bias relative to underlying layers. Preferred hardmask layers have high-silicon-content materials, preferably at least about 30% by weigh silicon, and more preferably from about 35% by weight to about 40% by weight silicon, based on the total weight of the hardmask layer. Suitable hardmask layers are commercially available and can be formed from a composition comprising a polymer or oligomer (e.g., silanes, siloxanes, silsesquioxanes, silicon oxynitride, silicon nitride, polysilicon, amorphous silicon, and combinations thereof) dissolved or dispersed in a solvent system. Some preferred monomers or polymers for use in the hardmask layer are selected from the group containing phenethyltrimethoxysilane (“PETMS”), 2-(carbomethoxy)ethyltrimethoxysilane (“CMETMS”), tetraethoxysilane (“TEOS”), methyltrimethoxysilane, phenyltrimethoxysilane, methyltrimethoxysilane (“MTMS”), ethyltrimethoxysilane (“ETMS”), (3-glycidyoxypropyl)triethoxysilane, 2-(3,4-epoxycyclohexyl)ethyltrimethyoxysilane (“ECHTMS”), and mixtures thereof. Any optional ingredients (e.g., surfactants, acid catalysts, base catalysts, and/or crosslinkers) are dissolved in the solvent system along with the polymer, monomer, and/or oligomer. Preferred hardmask compositions will preferably have a solids content of about 0.1% to about 70%, more preferably about 0.5% to about 10%, and even more preferably about 0.5% to about 1% by weight, based upon the total weight of the composition taken as 100% by weight.

After the hardmask composition is applied, it is preferably heated to a temperature of about 100° C. to about 300° C., and more preferably about 150° C. to about 250° C., and for a time period of about 30 seconds to about 120 seconds, and preferably about 45 seconds to about 60 seconds, to evaporate solvents. The average thickness (measured by ellipsometry over five locations and averaged) of the hardmask layer after baking is preferably about 5 nm to about 50,000 nm, more preferably about 5 nm to about 1000 nm, and even more preferably about 10 nm to about 30 nm.

After the hardmask composition is applied, an antireflective or adhesion layer comprising the functionalized polymers or monomers described above can be formed by any known application method, with one preferred method being spin-coating at speeds of about 500 rpm to about 3,000 rpm, and preferably about 1,200 rpm to about 2,000 rpm, for a time period of about 10 seconds to about 90 seconds, and preferably about 30 seconds to 60 seconds. Preferably, the antireflective or adhesion layer has good spin bowl compatibility, so that it will not react or form a precipitate with common photoresist solvents such as PGME, PGMEA, ethyl lactate, cyclohexanone, or mixtures thereof.

After the antireflective, adhesion, or assist layer composition is applied, it is preferably heated to a temperature of about 110° C. to about 250° C., and more preferably about 160° C. to about 180° C., for about 10 seconds to about 120 seconds, and preferably about 30 seconds to about 60 seconds, to evaporate solvents and crosslink the layer.

In another embodiment, the antireflective, adhesion or assist layer composition can be crosslinked by exposure to radiation. The antireflective, adhesion, or assist layer is subsequently fully or partially (e.g. in a pattern) exposed to radiation for a dose of about 10 mJ/cm² to about 2,000 mJ/cm², preferably about 80 mJ/cm² to about 1,000 mJ/cm², and more preferably about 140 mJ/cm² to about 300 mJ/cm² at a wavelength of from about 170 nm to about 365 nm, preferably from about 172 nm to about 365 nm.

The average thickness of the antireflective, adhesion, or assist layer after baking is preferably about 1 nm to about 2,000 nm, more preferably about 5 nm to about 80 nm, and even more preferably about 5 nm to about 40 nm. The average thickness is determined by taking the average of thickness measurements at five different locations of the antireflective or adhesion layer, with those thickness measurements being obtained using ellipsometry.

In some embodiments, the underlayers preferably possess light absorbing properties. For example, the refractive index (n value) of a cured antireflective layer at 193 nm or 248 nm will be at least about 1.2, preferably from about 1.3 to about 2, and more preferably from about 1.4 to about 1.8. The anti-reflective layers have an extinction coefficient (k value) of at least about 0.001, preferably from about 0.01 to about 0.8, and more preferably from about 0.05 to about 0.6, at the wavelength of use (e.g., 193 nm, 248 nm, or 365 nm). By varying the refractive index, extinction coefficient and thickness of the layers, a suitable combination can be found to lower the stack reflectivity to <1%.

Next, a photoresist (i.e., imaging layer) can be applied to the antireflective, adhesion, or assist layer, or to any intermediate layer on the antireflective, adhesion, or assist layer, to form a photoresist layer. The photoresist layer can be formed by any conventional method, with one preferred method being spin coating the photoresist composition at speeds of about 350 rpm to about 4,000 rpm (preferably about 1,000 rpm to about 2,500 rpm) for a time period of about 10 seconds to about 60 seconds (preferably about 10 seconds to about 30 seconds). The photoresist layer is then optionally post-application baked (“PAB”) at a temperature of at least about 70° C., preferably about 80° C. to about 150° C., and more preferably about 100° C. to about 150° C., for time periods of about 30 seconds to about 120 seconds. The average thickness (determined as described previously) of the photoresist layer after baking will typically be about 5 nm to about 120 nm, preferably about 10 nm to about 50 nm, and more preferably about 20 nm to about 40 nm.

The photoresist layer is subsequently patterned by exposure to radiation for a dose of about 10 mJ/cm² to about 200 mJ/cm², preferably about 15 mJ/cm² to about 100 mJ/cm², and more preferably about 20 mJ/cm² to about 80 mJ/cm² at a wavelength of from about 13.5 nm to about 365 nm, preferably from about 13.5 nm to about 248 nm. Suitable wavelengths include DUV, i-line, ArF, KrF, and EUV wavelengths. More specifically, the photoresist layer is exposed using a mask positioned above the surface of the photoresist layer. The mask has areas designed to permit the radiation to reflect from or pass through the mask and contact the surface of the photoresist layer. The remaining portions of the mask are designed to absorb the light to prevent the radiation from contacting the surface of the photoresist layer in certain areas. Those skilled in the art will readily understand that the arrangement of reflecting and absorbing portions is designed based upon the desired pattern to be formed in the photoresist layer and ultimately in the substrate or any intermediate layers.

After exposure, the photoresist layer is preferably subjected to a post-exposure bake (“PEB”) at a temperature of less than about 180° C., preferably about 60° C. to about 140° C., and more preferably about 80° C. to about 130° C., for a time period of about 30 seconds to about 120 seconds (preferably about 30 seconds to about 90 seconds).

The photoresist layer is then contacted with a developer to form the pattern. Depending upon whether the photoresist used is positive-working or negative-working, the developer will either remove the exposed portions of the photoresist layer or remove the unexposed portions of the photoresist layer to form the pattern. The pattern is then transferred through the various layers, and finally to the substrate. This pattern transfer can take place via plasma etching (e.g., CF₄ etchant, 02 etchant) or a wet etching or developing process.

Additional advantages of the various embodiments will be apparent to those skilled in the art upon review of the disclosure herein and the working examples below. It will be appreciated that the various embodiments described herein are not necessarily mutually exclusive unless otherwise indicated herein. For example, a feature described or depicted in one embodiment may also be included in other embodiments but is not necessarily included. Thus, the present disclosure encompasses a variety of combinations and/or integrations of the specific embodiments described herein.

As used herein, the phrase “and/or,” when used in a list of two or more items, means that any one of the listed items can be employed by itself or any combination of two or more of the listed items can be employed. For example, if a composition is described as containing or excluding components A, B, and/or C, the composition can contain or exclude A alone; B alone; C alone; A and B in combination; A and C in combination; B and C in combination; or A, B, and C in combination. The present description also uses numerical ranges to quantify certain parameters relating to various embodiments. It should be understood that when numerical ranges are provided, such ranges are to be construed as providing literal support for claim limitations that only recite the lower value of the range as well as claim limitations that only recite the upper value of the range. For example, a disclosed numerical range of about 10 to about 100 provides literal support for a claim reciting “greater than about 10” (with no upper bounds) and a claim reciting “less than about 100” (with no lower bounds).

### EXAMPLES

The following examples set forth methods in accordance with the disclosure. It is to be understood, however, that these examples are provided by way of illustration, and nothing therein should be taken as a limitation upon the overall scope.

### Example 1

### Grafting of Propiolic Acid to Bisphenol A Diglycidyl Ether

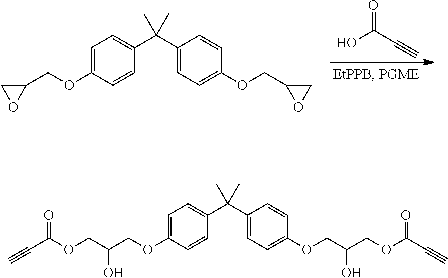

In this Example, 5.11 grams of diglycidyl ether bisphenol A (Sigma-Aldrich, St. Louis, MO), 2.10 grams of propiolic acid (Alfa Aesar, Tewksbury, MA), 0.278 gram of ethyltriphenylphosphonium bromide (EtPPB) (Sigma-Aldrich, St. Louis, MO), and 29.94 grams of propylene glycol monomethyl ether (PGME) (Fujifilm Ultrapure Solutions, Carrolton, TX) were added to a round-bottom flask and heated at 60° C. for 24 hours. The reaction was cooled to room temperature and recovered as a mother liquor.

### Example 2

### Grafting of Propiolic Acid to Tris(4-hydroxyphenyl)methane Triglycidyl Ether

In this Example, 6.91 grams of tris(4-hydroxylphenyl)methane triglycidyl ether (Sigma-Aldrich, St. Louis, MO), 3.15 grams of propiolic acid (Alfa Aesar, Tewksbury, MA), 0.139 gram of ethyltriphenylphosphonium bromide (EtPPB) (Sigma-Aldrich, St. Louis, MO), and 40.80 grams of propylene glycol monomethyl ether (PGME) (Fujifilm Ultrapure Solutions, Carrolton, TX) were added to a round-bottom flask and heated at 60° C. for 24 hours. The reaction was cooled to room temperature and recovered as a mother liquor.

### Example 3

### Grafting of Propiolic Acid to Tris(2,3-epoxypropyl) Isocyanurate

In this Example, 5.95 grams of tris(2,3-epoxypropyl) isocyanurate (TEPIC) (Sigma-Aldrich, St. Louis, MO), 4.20 grams of propiolic acid (Alfa Aesar, Tewksbury, MA), 0.140 gram of ethyltriphenylphosphonium bromide (EtPPB) (Sigma-Aldrich, St. Louis, MO), 15.44 grams of propylene glycol monomethyl ether (PGME) (Fujifilm Ultrapure Solutions, Carrolton, TX), and 15.44 grams of propylene glycol monomethyl ether acetate (PGMEA) (Fujifilm Ultrapure Solutions, Carrolton, TX) were added to a round-bottom flask and heated at 60° C. for 24 hours. The reaction was cooled to room temperature and recovered as a mother liquor.

### Example 4

### Grafting of Propiolic Acid to 9,9-Bis(4-hydroxyphenyl)fluorene Diglycidyl Ether

In this Example, 6.94 grams of 9,9-bis(4-hydroxyphenyl)fluorene diglycidyl ether (PDGE) (TCI Chemicals, Portland, OR), 2.1 grams of propiolic acid (Alfa Aesar, Tewksbury, MA), 0.278 gram of ethyltriphenylphosphonium bromide (EtPPB) (Sigma-Aldrich, St. Louis, MO), 13.98 grams of propylene glycol monomethyl ether (PGME) (Fujifilm Ultrapure Solutions, Carrolton, TX), and 13.98 grams of propylene glycol monomethyl ether acetate (PGMEA) (Fujifilm Ultrapure Solutions, Carrolton, TX) were added to a round-bottom flask and heated at 60° C. for 24 hours. The reaction was cooled to room temperature and recovered as a mother liquor.

### Example 5

### Grafting of Propiolic Acid to Epoxy Cresol Novolac

In this Example, 2.85 grams of epoxy cresol novolac (ECN) (Kukdo Chemical Co., Ltd., Seoul, Korea), 1.05 grams of propiolic acid (Alfa Aesar, Tewksbury, MA), 0.171 gram of ethyltriphenylphosphonium bromide (EtPPB) (Sigma-Aldrich, St. Louis, MO), and 6.29 grams of propylene glycol monomethyl ether (PGME) (Fujifilm Ultrapure Solutions, Carrolton, TX) were added to a round-bottom flask and heated at 60° C. for 24 hours. The reaction was cooled to room temperature and recovered as a mother liquor.

### Example 6

### Synthesis of Polymer 1

In this Example, 7.61 grams of methyl methacrylate (TCI Chemicals, Portland, OR), 0.53 gram of benzyl methacrylate (TCI Chemicals, Portland, OR), 0.11 gram of 4-methylstyrene (TCI Chemicals, Portland, OR), 2.13 grams of glycidyl methacrylate (Monomer Polymer and Dajac Labs, Ambler, PA), 0.673 gram of 4-cyano-4-(((dodecylthio)carbonothioyl)thio)pentanoic acid (BM1432) (Boron Molecular, Raleigh, NC), 0.0274 gram of azobisisobutyronitrile (AIBN) (Charkit, Norwalk, CT), and 21.96 grams of propylene glycol monomethyl ether acetate (PGMEA) (Fujifilm Ultrapure Solutions, Carrolton, TX) were added to a round-bottom flask and sparged with nitrogen for 10 minutes. The flask was then placed in an oil bath and heated at 70° C. for 24 hours. The reaction was cooled to room temperature and recovered as a mother liquor.

### Example 7

### Grafting of Propiolic Acid to Polymer 1 from Example 6

In this Example, 16.47 grams of the Polymer 1 mother liquor as synthesized in Example 6, 0.056 gram of ethyltriphenylphosphonium bromide (EtPPB) (Sigma-Aldrich, St. Louis, MO), 0.52 gram of propiolic acid (Alfa Aesar, Tewksbury, MA), and 10.98 grams of propylene glycol monomethyl ether (PGME) (Fujifilm Ultrapure Solutions, Carrolton, TX) were added to a round bottom flask and stirred at 60° C. for 24 hours before being cooled to room temperature and stored as a mother liquor.

### Example 8

### Synthesis of Polymer 2

In this Example, 3.5 grams of methyl methacrylate, 3.52 grams of benzyl methacrylate (TCI Chemicals, Portland, OR), 2.95 grams of 4-methylstyrene (TCI Chemicals, Portland, OR), 2.84 grams of glycidyl methacrylate (Monomer Polymer and Dajac Labs, Ambler, PA), 0.673 gram of 4-cyano-4-(((dodecylthio)carbonothioyl)thio)pentanoic acid (BM1432) (Boron Molecular, Raleigh, NC), 0.0274 gram of azobisisobutyronitrile (AIBN) (Charkit, Norwalk, CT), and 25.65 grams of propylene glycol monomethyl ether acetate (PGMEA) (Fujifilm Ultrapure Solutions, Carrolton, TX) were added to a round-bottom flask and sparged with nitrogen for 10 minutes. The flask was then placed in an oil bath and heated at 85° C. for 24 hours. The reaction was cooled to room temperature and recovered as a mother liquor.

### Example 9

### Grafting of Propiolic Acid to Polymer 2 from Example 8

In this Example, 15.39 grams of the Polymer 2 mother liquor as synthesized in Example 8, 0.059 gram of ethyltriphenylphosphonium bromide (EtPPB) (Sigma-Aldrich, St. Louis, MO), 0.56 gram of propiolic acid (Alfa Aesar, Tewksbury, MA), and 5.13 grams of propylene glycol monomethyl ether (PGME) (Fujifilm Ultrapure Solutions, Carrolton, TX) were added to a round bottom flask and stirred at 60° C. for 24 hours before being cooled to room temperature and stored as a mother liquor.

### Example 10

### Synthesis of Polymer 3

In this Example, 4.59 grams of 2-isopropyl-2-adamantyl methacrylate (TCI Chemicals, Portland, OR), 4.62 grams of glycidyl methacrylate (Monomer Polymer and Dajac Labs, Ambler, PA), 0.336 gram of 4-cyano-4-(((dodecylthio)carbonothioyl)thio)pentanoic acid (BM1432) (Boron Molecular, Raleigh, NC), 0.0137 gram of azobisisobutyronitrile (AIBN) (Charkit, Norwalk, CT), and 18.42 grams of propylene glycol monomethyl ether acetate (PGMEA) (Fujifilm Ultrapure Solutions, Carrolton, TX) were added to a round-bottom flask and sparged with nitrogen for 10 minutes. The flask was then placed in an oil bath and heated at 70° C. for 24 hours. The reaction was cooled to room temperature and recovered as a mother liquor.

### Example 11

### Grafting of Propiolic Acid to Polymer 3 from Example 10

In this Example, 11.05 grams of the Polymer 3 mother liquor as synthesized in Example 9, 0.097 gram of ethyltriphenylphosphonium bromide (EtPPB) (Sigma-Aldrich, St. Louis, MO), 0.91 gram of propiolic acid (Alfa Aesar, Tewksbury, MA), and 3.68 grams of propylene glycol monomethyl ether (PGME) (Fujifilm Ultrapure Solutions, Carrolton, TX) were added to a round bottom flask and stirred at 60° C. for 24 hours before being cooled to room temperature and stored as a mother liquor.

### Example 12

### Synthesis of Polymer 4

In this Example, 28.43 grams of glycidyl methacrylate (Monomer Polymer and Dajac Labs, Ambler, PA), 1.34 grams of 4-cyano-4-(((dodecylthio)carbonothioyl)thio)pentanoic acid (BM1432) (Boron Molecular, Raleigh, NC), 0.0547 gram of azobisisobutyronitrile (AIBN) (Charkit, Norwalk, CT), and 56.86 grams of propylene glycol monomethyl ether acetate (PGMEA) (Fujifilm Ultrapure Solutions, Carrolton, TX) were added to a round-bottom flask and sparged with nitrogen for 10 minutes. The flask was then placed in an oil bath and heated at 70° C. for 24 hours. The reaction was cooled to room temperature and recovered as a mother liquor.

### Example 13

### Synthesis of Polymer 5

In this Example, 95.11 grams of methyl methacrylate (TCI chemicals, Portland, OR), 7.11 grams of glycidyl methacrylate (Monomer Polymer and Dajac Labs, Ambler, PA), 7.76 grams of 4-cyano-4-(((dodecylthio)carbonothioyl)thio) pentanoic acid (Boron Molecular, Raleigh, NC), 0.316 gram of 2,2′-azobis(2-methylpropionitrile) (AIBN) (Charkit, Norwalk, CT) and 204.44 grams of PGMEA (Fujifilm Ultra Pure Solutions, Inc., Carrollton, TX) were added to a round bottom flask and sparged with N₂ for 10 minutes. The reaction was held at 70° C. under nitrogen with magnetic stirring for 24 hours.

### Example 14

### Grafting of Propiolic Acid to Polymer 5 from Example 13

In this Example, 30.67 grams of the Polymer 5 mother liquor as synthesized in Example 13, 0.093 gram of ethyltriphenylphosphonium bromide (EtPPB) (Sigma-Aldrich, St. Louis, MO), 0.35 gram of propiolic acid (Alfa Aesar, Tewksbury, MA), and 20.44 grams of propylene glycol monomethyl ether (PGME) (Fujifilm Ultrapure Solutions, Carrolton, TX) were added to a round bottom flask and stirred at 60° C. for 24 hours before being cooled to room temperature and stored as a mother liquor.

### Example 15

### Synthesis of Polymer 6

In this Example, 9.01 grams of methyl methacrylate (TCI chemicals, Portland, OR), 1.42 grams of glycidyl methacrylate (Monomer Polymer and Dajac Labs, Ambler, PA), 0.673 grams of 4-cyano-4-(((dodecylthio)carbonothioyl)thio) pentanoic acid (Boron Molecular, Raleigh, NC), 0.027 gram of 2,2′-azobis(2-methylpropionitrile) (AIBN) (Charkit, Norwalk, CT), and 20.86 grams of PGMEA (Fujifilm Ultra Pure Solutions, Inc., Carrollton, TX) were added to a round bottom flask and sparged with N₂ for 10 minutes. The reaction was held at 70° C. under nitrogen with magnetic stirring for 24 hours.

### Example 16

### Grafting of Propiolic Acid to Polymer 6 from Example 15

In this Example, 15.64 grams of the Polymer 5 mother liquor as synthesized in Example 15, 0.093 gram of ethyltriphenylphosphonium bromide (EtPPB) (Sigma-Aldrich, St. Louis, MO), 0.35 gram of propiolic acid (Alfa Aesar, Tewksbury, MA), and 10.43 grams of propylene glycol monomethyl ether (PGME) (Fujifilm Ultrapure Solutions, Carrolton, TX) were added to a round bottom flask and stirred at 60° C. for 24 hours before being cooled to room temperature and stored as a mother liquor.

### Example 17

### Grafting of 9-ACA, Pentafluorobenzoic acid, and Propiolic acid to Polymer 4 from Example 12

In this Example, 10.66 grams of the Polymer 4 mother liquor as synthesized in Example 11, 1.11 grams of 9-anthracenecarboxylic acid (Midori Kagaku, Tokyo, Japan), 2.54 grams of pentafluorobenzoic acid (TCI Chemicals, Portland, OR), 0.186 gram of ethyltriphenylphosphonium bromide (EtPPB) (Sigma-Aldrich, St. Louis, MO), 0.52 gram of propiolic acid (Alfa Aesar, Tewksbury, MA), and 7.92 grams of propylene glycol monomethyl ether (PGME) (Fujifilm Ultrapure Solutions, Carrolton, TX) were added to a round bottom flask and stirred at 60° C. for 24 hours before being cooled to room temperature and stored as a mother liquor.

### Example 18

### Grafting of Pentafluorobenzoic acid, 3,5-Diiodosalicylic acid, and Propiolic Acid to Polymer 4 from Example 12

In this Example, 10.66 grams of the Polymer 4 mother liquor as synthesized in Example 11, 2.12 grams of pentafluorobenzoic acid (TCI chemicals, Portland, OR), 2.92 grams of 3,5-diiodosalicylic acid (TCI Chemicals, Portland, OR), 0.186 gram of ethyltriphenylphosphonium bromide (EtPPB) (Sigma-Aldrich, St. Louis, MO), 0.52 gram of propiolic acid (Alfa Aesar, Tewksbury, MA), and 9.31 grams of propylene glycol monomethyl ether (PGME) (Fujifilm Ultrapure Solutions, Carrolton, TX) were added to a round bottom flask and stirred at 60° C. for 24 hours before being cooled to room temperature and stored as a mother liquor.

### Example 19

### Grafting of 3,5-Diiodosalicylic Acid and Propiolic Acid to Epoxy Cresol Novolac

In this Example, 3.8 grams of epoxy cresol novolac (ECN) (Kukdo Chemical Co., Ltd., Seoul, Korea), 4.68 grams of 3,5-diiodosalicylic acid (TCI chemicals, Portland, OR), 0.278 gram of ethyltriphenylphosphonium bromide (EtPPB) (Sigma-Aldrich, St. Louis, MO), 0.56 gram of propiolic acid (Alfa Aesar, Tewksbury, MA), and 13.98 grams of propylene glycol monomethyl ether (PGME) (Fujifilm Ultrapure Solutions, Carrolton, TX) were added to a round bottom flask and stirred at 60° C. for 24 hours before being cooled to room temperature and stored as a mother liquor.

### Example 20

### Synthesis of Polymer 7

In this Example, 5.51 grams of 4-(tert-butoxycarbonyloxy)styrene (Heraeus Epurio, Dayton, OH), 3.55 grams of glycidyl methacrylate (Monomer Polymer and Dajac Labs, Ambler, PA), 0.336 gram of 4-cyano-4-(((dodecylthio)carbonothioyl)thio)pentanoic acid (BM1432) (Boron Molecular, Raleigh, NC), 0.0137 gram of azobisisobutyronitrile (AIBN) (Charkit, Norwalk, CT), and 18.12 grams of propylene glycol monomethyl ether acetate (PGMEA) (Fujifilm Ultrapure Solutions, Carrolton, TX) were added to a round-bottom flask and sparged with nitrogen for 10 minutes. The flask was then placed in an oil bath and heated at 80° C. for 24 hours. The reaction was cooled to room temperature and recovered as a mother liquor.

### Example 21

### Grafting of Propiolic Acid to Polymer 7 from Example 20

In this Example, 24.46 grams of the Polymer 7 mother liquor as synthesized in Example 20, 0.167 grams of ethyltriphenylphosphonium bromide (EtPPB) (Sigma-Aldrich, St. Louis, MO), 1.58 grams of propiolic acid (Alfa Aesar, Tewksbury, MA), and 9.90 grams of propylene glycol monomethyl ether (PGME) (Fujifilm Ultrapure Solutions, Carrolton, TX) were added to a round bottom flask and stirred at 60° C. for 24 hours before being cooled to room temperature and stored as a mother liquor.

### Example 22

### Synthesis of Polymer 8

In this Example, 10.09 grams of cyclohexyl methacrylate (TCI Chemicals, Portland, OR), 2.84 grams of glycidyl methacrylate (Monomer Polymer and Dajac Labs, Ambler, PA), 0.538 gram of 4-cyano-4-(((dodecylthio)carbonothioyl)thio)pentanoic acid (BM1432) (Boron Molecular, Raleigh, NC), 0.0219 gram of azobisisobutyronitrile (AIBN) (Charkit, Norwalk, CT), and 26.99 grams of propylene glycol monomethyl ether acetate (PGMEA) (Fujifilm Ultrapure Solutions, Carrolton, TX), were added to a round-bottom flask and sparged with nitrogen for 10 minutes. The flask was then placed in an oil bath and heated at 70° C. for 24 hours. The reaction was cooled to room temperature and recovered as a mother liquor.

### Example 23

### Grafting of Propiolic Acid to Polymer 8 from Example 22

In this Example, 16.98 grams of the Polymer 8 mother liquor as synthesized in Example 22, 0.065 grams of ethyltriphenylphosphonium bromide (EtPPB) (Sigma-Aldrich, St. Louis, MO), 0.613 grams of propiolic acid (Alfa Aesar, Tewksbury, MA), and 6.34 grams of propylene glycol monomethyl ether (PGME) (Fujifilm Ultrapure Solutions, Carrolton, TX) were added to a round bottom flask and stirred at 60° C. for 24 hours before being cooled to room temperature and stored as a mother liquor.

### Example 24

### Formulation of Product from Example 1

In this Example, 5.00 grams of the mother liquor as functionalized in Example 1, 4 grams of PGME (Fujifilm Ultrapure Solutions, Carrolton, TX), and 11 grams of PGMEA (Fujifilm Ultrapure Solutions, Carrolton, TX) were added to a 100-mL Aicello bottle and mixed for 5 minutes. The resulting solution was filtered with a 0.1-μm PTFE endpoint filter.

### Example 25

### Formulation of Product from Example 5

In this Example, 2.50 grams of the mother liquor as functionalized in Example 5, 7.50 grams of PGME (Fujifilm Ultrapure Solutions, Carrolton, TX), and 11 grams of PGMEA (Fujifilm Ultrapure Solutions, Carrolton, TX) were added to a 100-mL Aicello bottle and mixed for 5 minutes. The resulting solution was filtered with a 0.1-μm PTFE endpoint filter.

### Example 26

### Formulation of Functionalized Polymer 1 from Example 7

In this Example, 6.00 grams of the Polymer 1 mother liquor as functionalized in Example 6, 60 grams of propylene glycol monomethyl ether acetate (PGMEA) (Fujifilm Ultrapure Solutions, Carrolton, TX), and 34 grams of propylene glycol monomethyl ether (PGME) (Fujifilm Ultrapure Solutions, Carrolton, TX) were added to a 100-mL Aicello bottle and mixed for 24 hours. The resulting solution was filtered with a 0.1-μm PTFE endpoint filter.

### Example 27

### Formulation of Functionalized Polymer 2 from Example 9

In this Example, 6.00 grams of the Polymer 2 mother liquor as functionalized in Example 8, 60 grams of propylene glycol monomethyl ether acetate (PGMEA) (Fujifilm Ultrapure Solutions, Carrolton, TX), and 34 grams of propylene glycol monomethyl ether (PGME) (Fujifilm Ultrapure Solutions, Carrolton, TX) were added to a 100-mL Aicello bottle and mixed for 24 hours. The resulting solution was filtered with a 0.1-μm PTFE endpoint filter.

### Example 28

### Formulation of Functionalized Polymer 3 from Example 11

In this Example, 0.8 gram of the Polymer 3 mother liquor as functionalized in Example 11, 65.2 grams of propylene glycol monomethyl ether acetate (PGMEA) (Fujifilm Ultrapure Solutions, Carrolton, TX), and 34 grams of propylene glycol monomethyl ether (PGME) (Fujifilm Ultrapure Solutions, Carrolton, TX) were added to a 100-mL Aicello bottle and mixed for 24 hours. The resulting solution was filtered with a 0.1-μm PTFE endpoint filter.

### Example 29

### Formulation of Functionalized Polymer 5 from Example 14

In this Example, 5.00 grams of the mother liquor as functionalized in Example 14, 7.5 grams of PGME (Fujifilm Ultrapure Solutions, Carrolton, TX), and 7.5 grams of PGMEA (Fujifilm Ultrapure Solutions, Carrolton, TX) were added to a 100-mL Aicello bottle and mixed for 5 minutes. The resulting solution was filtered with a 0.1-μm PTFE endpoint filter.

### Example 30

### Formulation of Functionalized Polymer 6 from Example 16

In this example, 5.00 grams of the mother liquor as functionalized in Example 16, 7.50 grams of PGME (Fujifilm Ultrapure Solutions, Carrolton, TX), and 7.50 grams of PGMEA (Fujifilm Ultrapure Solutions, Carrolton, TX) were added to a 100-mL Aicello bottle and mixed for 5 minutes. The resulting solution was filtered with a 0.1-μm PTFE endpoint filter.

### Example 31

### 193-nm Immersion Exposure

The material formulated in Example 27 was coated on a 300-mm silicon wafer by spin coating at 1,500 rpm for 60 seconds and then was baked at 205° C. for 60 seconds. The material formulated in Example 26 was coated on top of the layer of material from Example 27 and was baked at 205° C. for 60 seconds. AIM5484 photoresist from JSR was coated on top of the layer of material from Example 12 by spin coating at 1,185 rpm and baking at 120° C. for 60 seconds as a post-apply bake (PAB). The wafer was then exposed to varying doses of 193-nm radiation and then baked at 100° C. for 60 seconds before being developed by aqueous base to reveal the pattern. An SEM image of this wafer is shown in FIG. 1.

### Example 32

### EUV Exposure

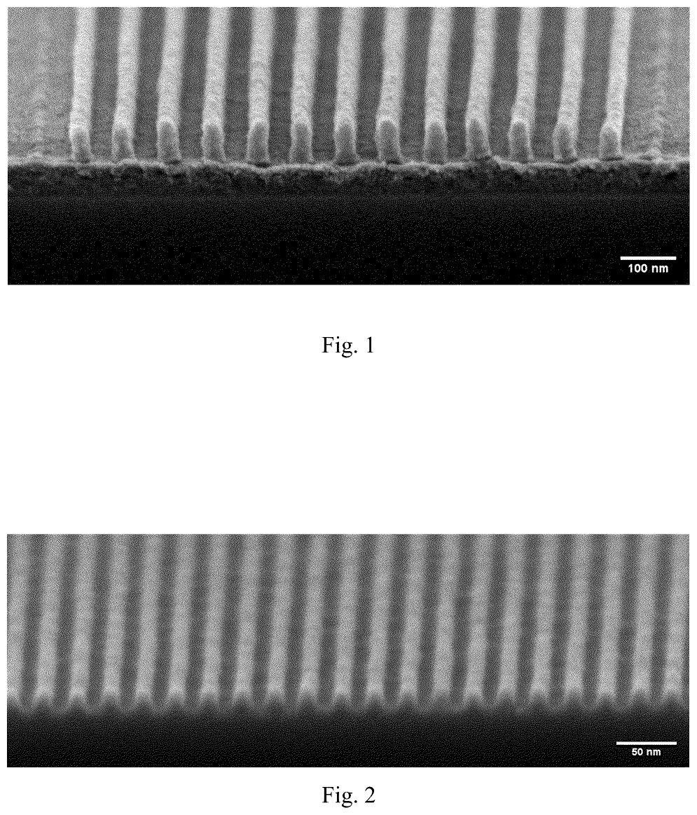

The material formulated in Example 28 was coated on a 300-mm silicon wafer by spin coating at 1,500 rpm for 60 seconds and then was baked at 160° C. for 60 seconds. Pentaline photoresist (TOK, Kangawa, Japan) was coated on top of the layer of material from Example 28 by spin coating at 1,800 rpm and baking at 110° C. for 60 seconds as a post-apply bake (PAB). The wafer was then exposed to varying doses of EUV radiation in an ASML NXE3400 scanner and then baked at 90° C. for 60 seconds before being developed by aqueous base to reveal the pattern. FIG. 2 provides an SEM image of this wafer.

### Example 33

### Crosslinking Testing

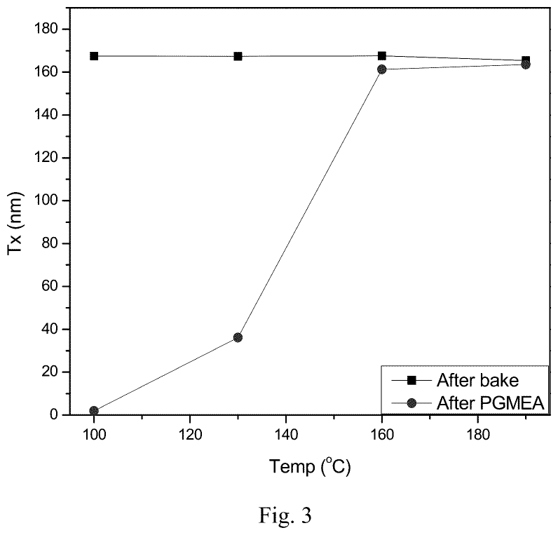

The material formulated in Example 24 was coated on multiple 100-mm silicon wafers by spin coating at 1,500 rpm for 60 seconds and then was baked at varying temperatures for 60 seconds. FIG. 3 shows the thickness of the films formed after baking and then after washing with PGMEA as a function of bake temperature.

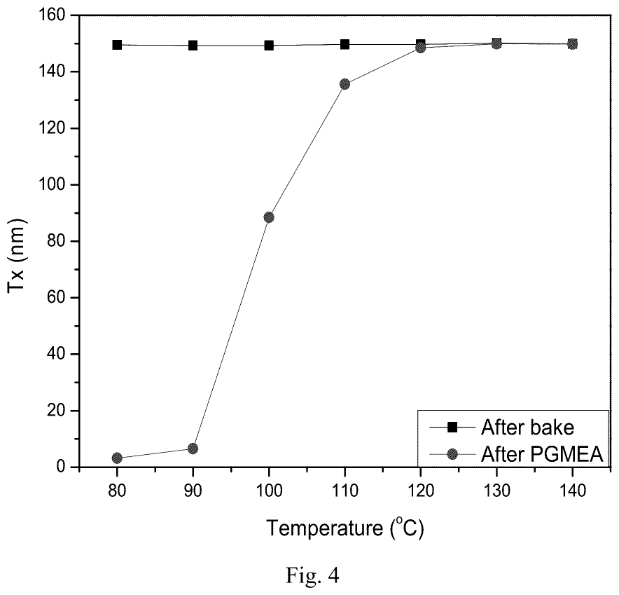

The material formulated in Example 25 was coated on multiple 100-mm silicon wafers by spin coating at 1,500 rpm for 60 seconds and then was baked at varying temperatures for 60 seconds. FIG. 4 shows the thickness of the films formed after baking and then after washing with PGMEA as a function of bake temperature.

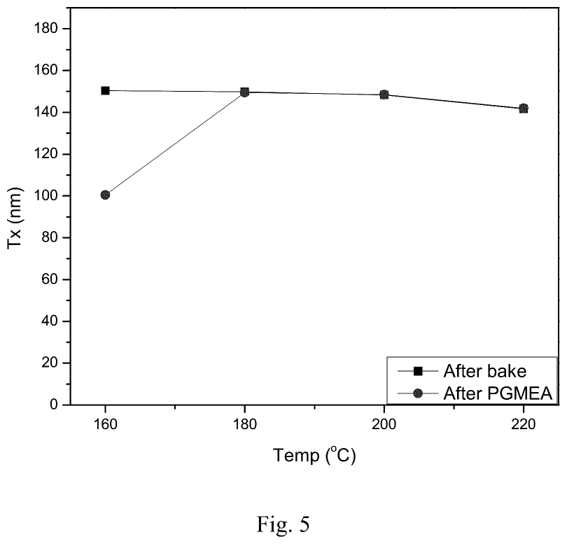

The material formulated in Example 29 was coated on multiple 100-mm silicon wafers by spin coating at 1,500 rpm for 60 seconds and then was baked at varying temperatures for 60 seconds. FIG. 5 shows the thickness of the films formed after baking and then after washing with PGMEA as a function of bake temperature.

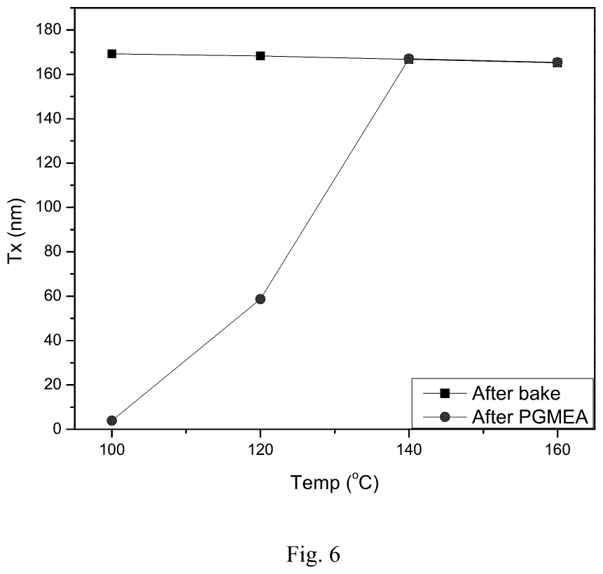

The material formulated in Example 30 was coated on multiple 100-mm silicon wafers by spin coating at 1,500 rpm for 60 seconds and then was baked at varying temperatures for 60 seconds. FIG. 6 shows the thickness of the films formed after baking and then after washing with PGMEA as a function of bake temperature.

### Example 34

### Photocrosslinking

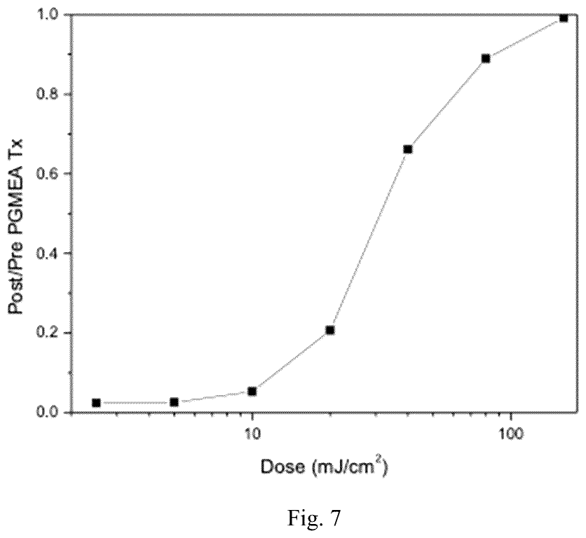

The material formulated in Example 25 was coated on a 100-mm silicon wafer by spin coating at 1,500 rpm for 60 seconds and was baked at 80° C. for 60 seconds. The film was then exposed to broadband UV radiation using an Oriel Solar Simulator at a variety of doses (dose measured at 254 nm) before being washed with PGMEA. The thickness before and after PGMEA strip was measured by ellipsometry and the ratio is shown in FIG. 7 as a function of dose.

1. A method of forming a structure, said method comprising:
forming an underlayer on a stack, said underlayer being formed from a composition comprising a compound comprising an acetylenic carboxylic acid reacted with an epoxy group;
optionally forming one or more intermediate layers on said underlayer, there being an uppermost intermediate layer on said substrate surface, if one or more intermediate layers are present; and
applying a photoresist layer to said one or more intermediate layers, if present, or to said underlayer, if no intermediate layers are present.

2. The method of claim 1, wherein said forming comprises applying said composition to said stack and subjecting said composition to one or both of heat or UV radiation to induce crosslinking of said compound.

3. The method of claim 1, wherein:
(a) said composition comprises less than about 0.1% by weight of a crosslinking agent, based on the weight of the composition taken as 100% by weight;
(b) said compound is self-crosslinking; or
(c) both (a) and (b) are true.

4. The method of claim 1, wherein said acetylenic carboxylic acid is chosen from C₃ to C₈ acetylenic carboxylic acids.

5. The method of claim 1, wherein said acetylenic carboxylic acid is chosen from propiolic acid, tetrolic acid, 4-pentynoic acid, 5-hexynoic acid, or mixtures thereof.

6. The method of claim 1, wherein said compound is chosen from monomeric compounds, oligomeric compounds, polymeric compounds, or mixtures thereof.

7. The method of claim 1, wherein said epoxy group is present as part of:
(i) a compound chosen from tris-(2,3-epoxy propyl)isocyanurate, bisphenol A diglycidyl ether, 9,9-bis(4-glycidyloxyphenyl)fluorene, tris-(4-hydroxyphenyl)methane triglycidyl ether, or mixtures thereof;
(ii) a polymer chosen from epoxy cresol novolac, poly(glycidyl methacrylate), epoxy phenyl novolac, poly(4-glycidyloxystyrene), bisphenol A novolac epoxy resin, poly(glycidyl acrylate), or mixtures thereof; or
(iii) combinations of (i) and (ii).

8. The method of claim 1, wherein said compound is chosen from one or more of

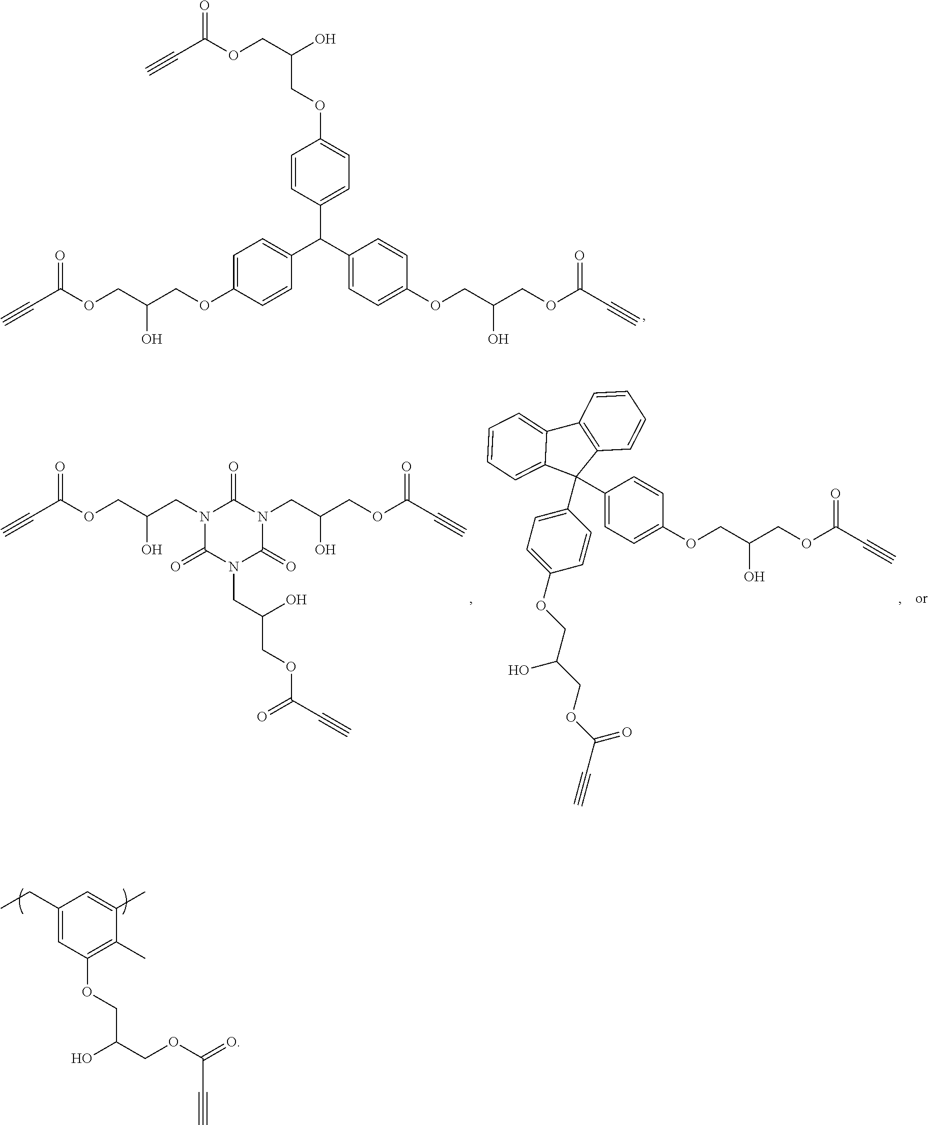

9. The method of claim 1, said stack comprising:
a substrate having a surface; and
optionally one or more additional intermediate layers on said surface, there being an uppermost additional intermediate layer on said surface, if one or more additional intermediate layers are present, said underlayer being on said uppermost additional intermediate layer, if present, or on said surface, if no additional intermediate layers are present.

10. The method of claim 9, said substrate comprising silicon, SiGe, SiO₂, Si₃N₄, SiON, SiCO:H, tetramethyl silate and tetramethylcyclotetrasiloxane combinations, aluminum, tungsten, tungsten silicide, gallium arsenide, germanium, tantalum, tantalum nitride, Ti₃N₄, hafnium, HfO₂, ruthenium, indium phosphide, or glass.

11. The method of claim 1, further comprising:
exposing said photoresist layer to radiation;
forming a pattern in said photoresist layer after said subjecting said photoresist layer to radiation; and
transferring said pattern to said underlayer.

12. The method of claim 1, wherein said underlayer comprises an anti-reflective layer, a carbon-rich layer, or an adhesion layer.

13. A structure comprising:
a substrate having a surface;
optionally one or more intermediate layers on said substrate surface, there being an uppermost intermediate layer on said substrate surface, if one or more intermediate layers are present;
an underlayer on said substrate surface, or on said uppermost intermediate layer, if present, said underlayer comprising a compound comprising an acetylenic carboxylic acid reacted with an epoxy group; and
a photoresist on said underlayer.

14. The structure of claim 13, wherein said acetylenic carboxylic acid is chosen from C₃ to C₈ acetylenic carboxylic acids.

15. The structure of claim 13, wherein said acetylenic carboxylic acid is chosen from propiolic acid, tetrolic acid, 4-pentynoic acid, 5-hexynoic acid, or mixtures thereof.

16. The structure of claim 13, wherein said compound is chosen from monomeric compounds, oligomeric compounds, polymeric compounds, or mixtures thereof.

17. The structure of claim 13, wherein said epoxy group is present as part of:
(i) a compound chosen from tris-(2,3-epoxy propyl)isocyanurate, bisphenol A diglycidyl ether, 9,9-bis(4-glycidyloxyphenyl)fluorene, tris-(4-hydroxyphenyl)methane triglycidyl ether, or mixtures thereof;
(ii) a polymer chosen from epoxy cresol novolac, poly(glycidyl methacrylate), epoxy phenyl novolac, poly(4-glycidyloxystyrene), bisphenol A novolac epoxy, poly(glycidyl acrylate), or mixtures thereof; or
(iii) combinations of (i) and (ii).

18. The structure of claim 13, wherein said compound is chosen from one more of

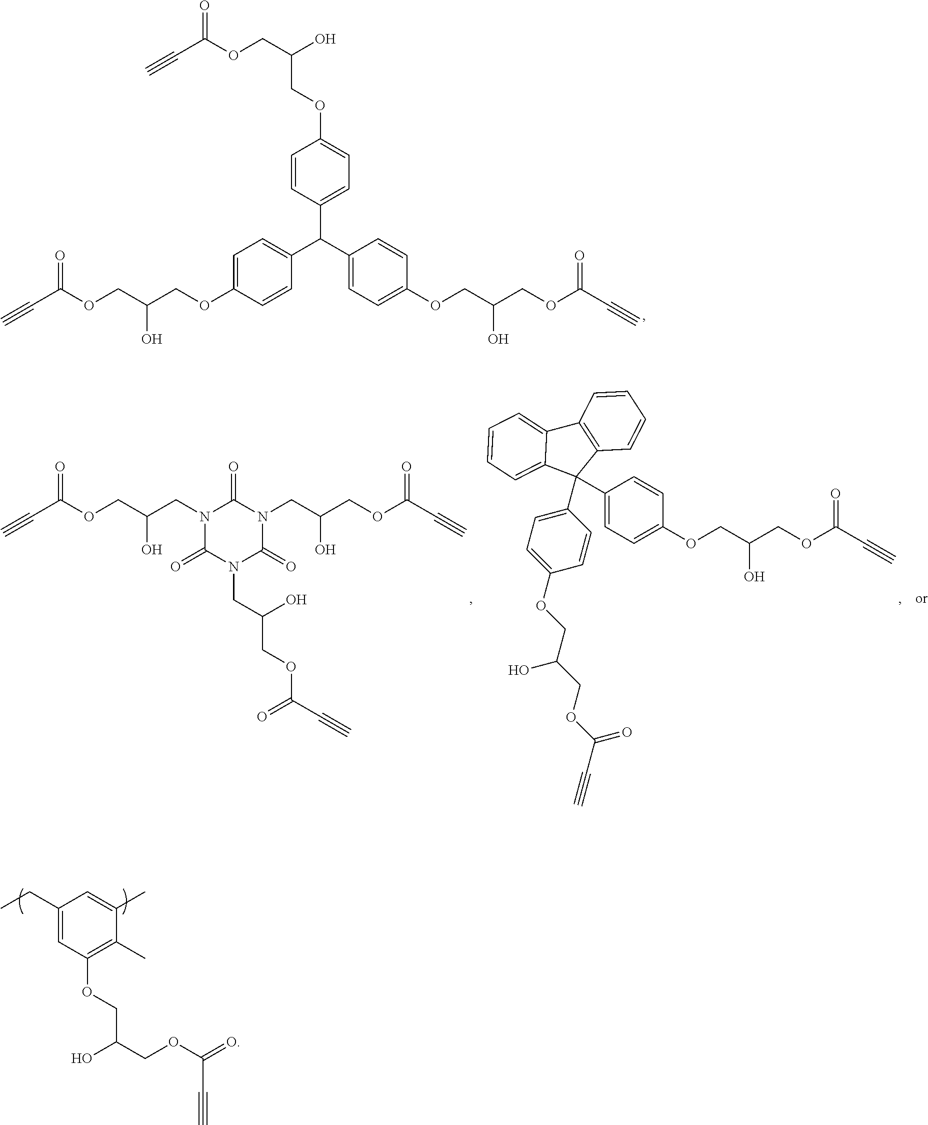

19. The structure of claim 13, wherein said underlayer comprises an anti-reflective layer, a carbon-rich layer, or an adhesion layer.

20. The structure of claim 13, said substrate comprising silicon, SiGe, SiO₂, Si₃N₄, SiON, SiCO:H, tetramethyl silate and tetramethylcyclotetrasiloxane combinations, aluminum, tungsten, tungsten silicide, gallium arsenide, germanium, tantalum, tantalum nitride, Ti₃N₄, hafnium, HfO₂, ruthenium, indium phosphide, or glass.

21. A method comprising reacting an acetylenic carboxylic acid with an epoxy group at a temperature of about 100° C. or lower to form a reaction product.

22. The method of claim 21, wherein said reacting is carried out in a reaction solution having a molar ratio of acetylenic carboxylic acid to epoxy group of about 0.8:1 to about 1.2:1.

23. The method of claim 21, wherein said reacting is carried out at a temperature of 50° C. to about 100° C.

24. The method of claim 21, wherein said reacting is carried out for about 4 hours to about 36 hours.

25. The method of claim 21, wherein said acetylenic carboxylic acid is chosen from C₃ to C₈ acetylenic carboxylic acids.

26. The method of claim 21, wherein said acetylenic carboxylic acid is chosen from propiolic acid, tetrolic acid, 4-pentynoic acid, 5-hexynoic acid, or mixtures thereof.

27. The method of claim 21, wherein said epoxy group is present as part of a monomeric compound, an oligomeric compound, a polymeric compound, or two or more of the foregoing.

28. The method of claim 21, wherein said epoxy group is present as part of:
(i) a compound chosen from tris-(2,3-epoxy propyl)isocyanurate, bisphenol A diglycidyl ether, 9,9-bis(4-glycidyloxyphenyl)fluorene, tris-(4-hydroxyphenyl)methane triglycidyl ether, or mixtures thereof;
(ii) a polymer chosen from epoxy cresol novolac, poly(glycidyl methacrylate), epoxy phenyl novolac, poly(4-glycidyloxystyrene), bisphenol A novolac epoxy, poly(glycidyl acrylate), or mixtures thereof; or
(iii) combinations of (i) and (ii).

29. The method of claim 21, wherein said reaction product is chosen from one more of

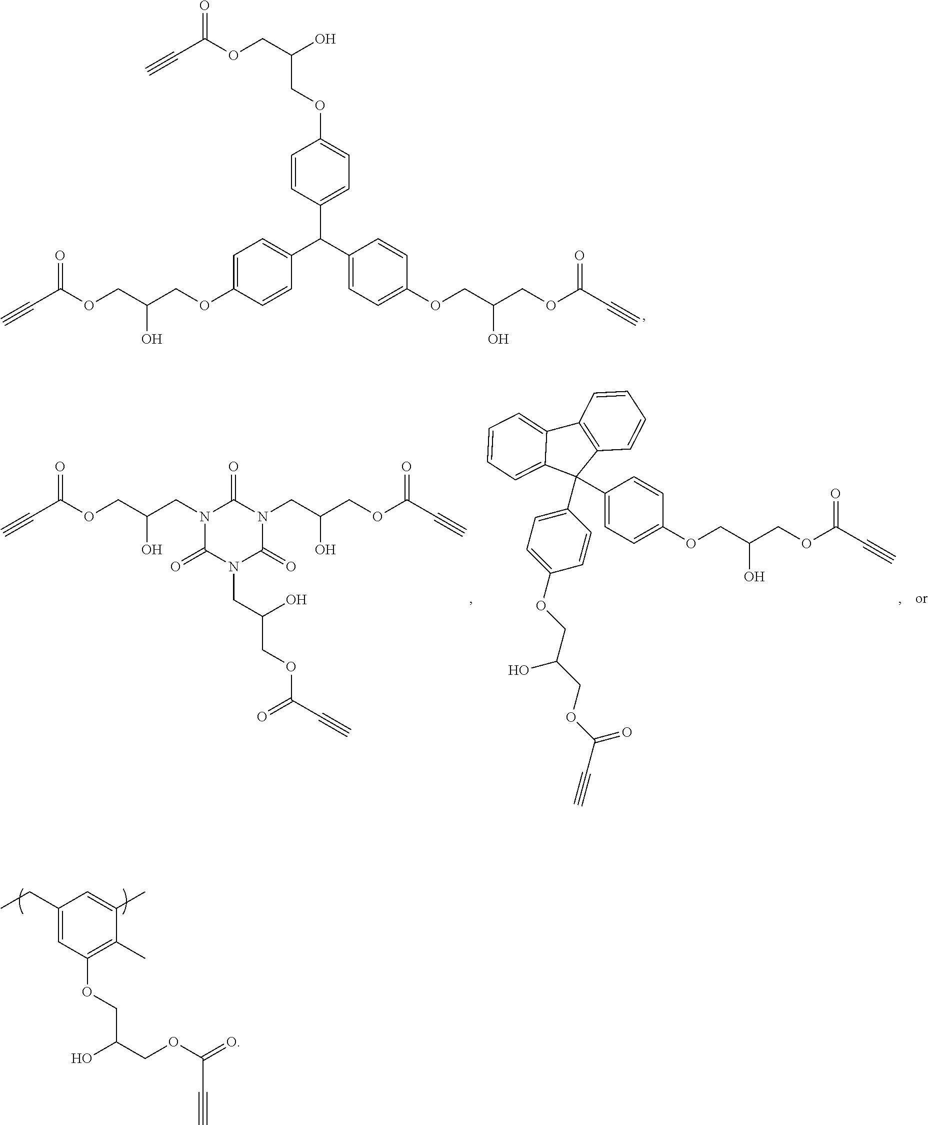

30. A compound having a formula chosen from

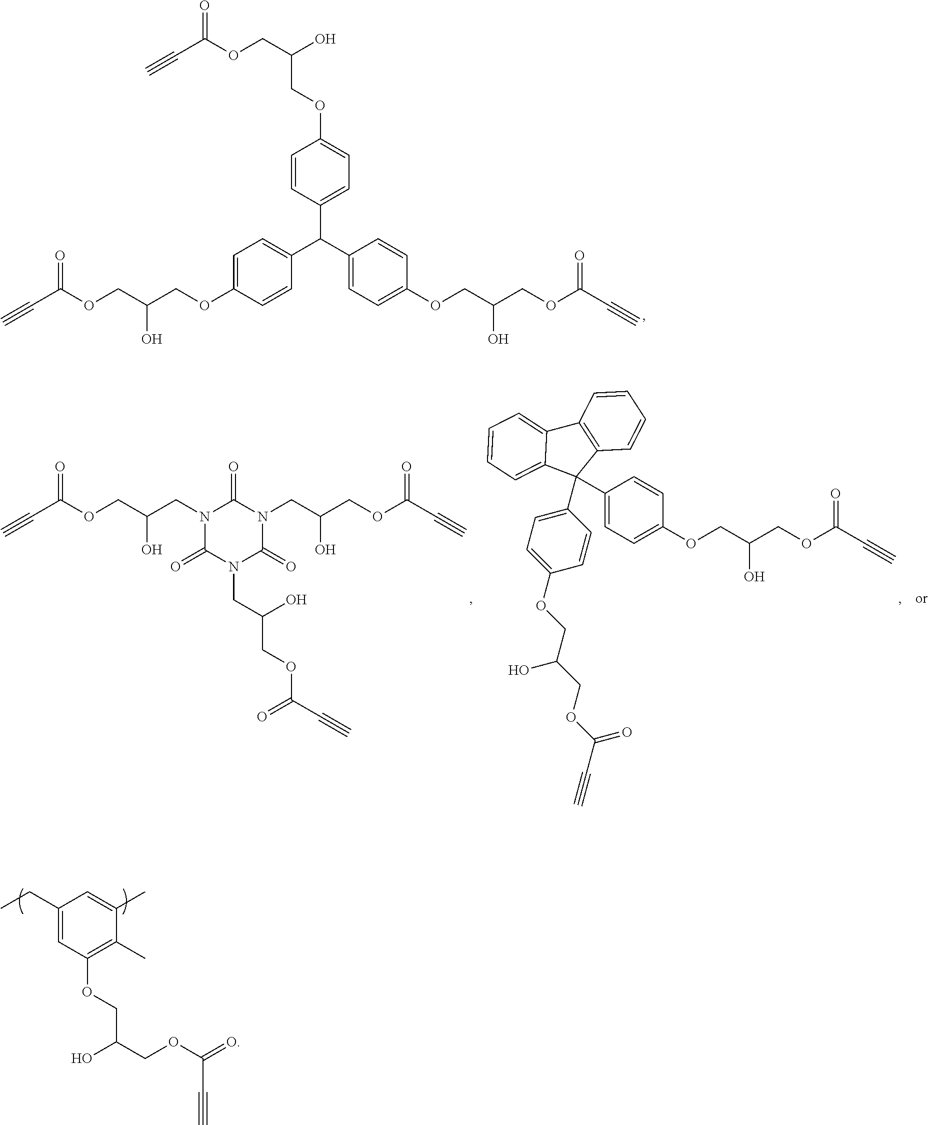

31. A crosslinked layer comprising an acetylenic carboxylic acid reacted with an epoxy group.

32. The crosslinked layer of claim 31, wherein said acetylenic carboxylic acid is chosen from C₃ to C₈ acetylenic carboxylic acids.

33. The crosslinked layer of claim 31, wherein said acetylenic carboxylic acid is chosen from propiolic acid, tetrolic acid, 4-pentynoic acid, 5-hexynoic acid, or mixtures thereof.

34. The crosslinked layer of claim 31, wherein said epoxy group is present as part of a monomeric compound, an oligomeric compound, a polymeric compound, or two or more of the foregoing.

35. The crosslinked layer of claim 31, wherein said epoxy group is present as part of:
(i) a compound chosen from tris-(2,3-epoxy propyl)isocyanurate, bisphenol A diglycidyl ether, 9,9-bis(4-glycidyloxyphenyl)fluorene, tris-(4-hydroxyphenyl)methane triglycidyl ether, or mixtures thereof;
(ii) a polymer chosen from epoxy cresol novolac, poly(glycidyl methacrylate), epoxy phenyl novolac, poly(4-glycidyloxystyrene), bisphenol A novolac epoxy resin, poly(glycidyl acrylate), or mixtures thereof; or
(iii) combinations of (i) and (ii).

36. The crosslinked layer of claim 31, wherein said compound is chosen from one more of

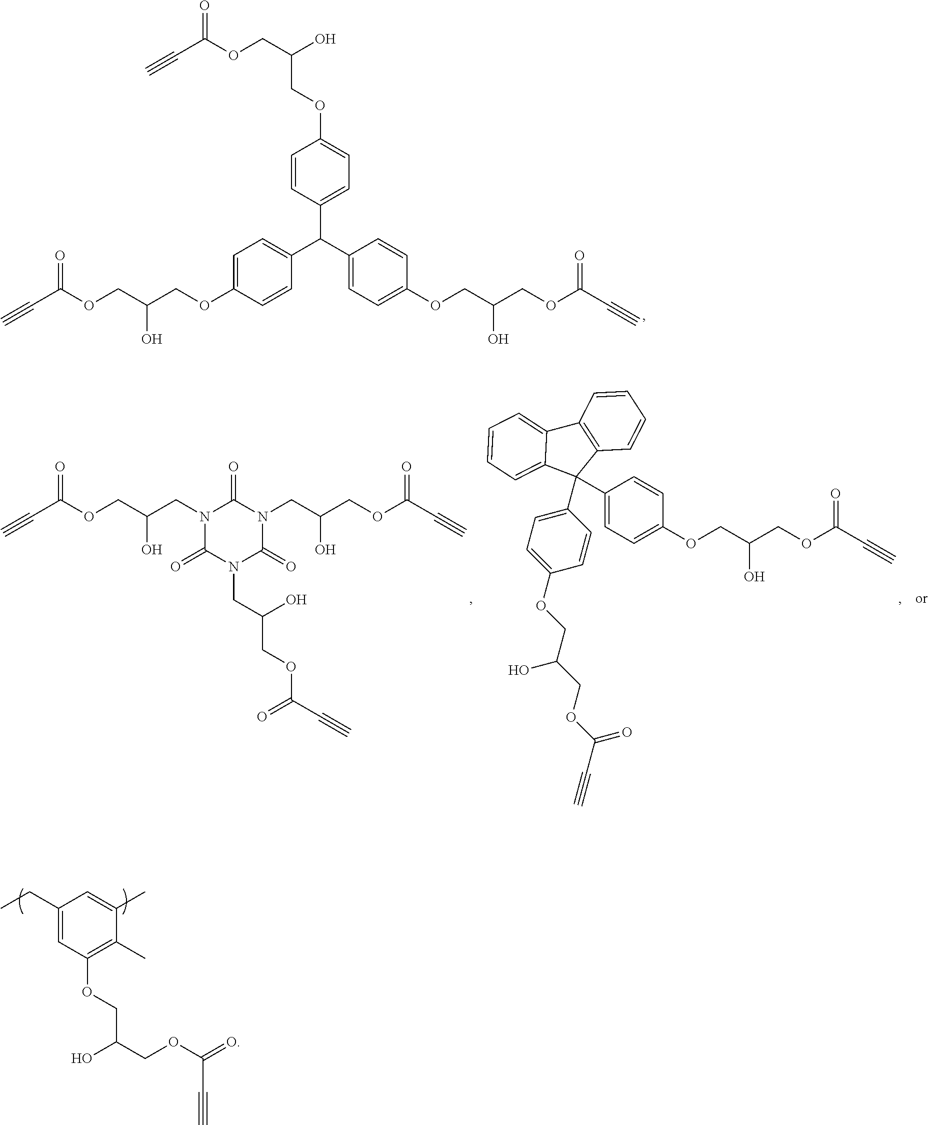

37. The crosslinked layer of claim 31, wherein said compound comprises a moiety having the structure 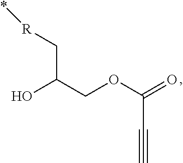
where * represents an attachment point of said moiety to a remainder of said compound, and R is chosen from oxygen, carbon, or nitrogen.
